(19) United States
(12) Patent Application Publication
(10) Pub. No.: US 2017/0189591 A1
(43) Pub. Date: Jul. 6, 2017

(54) WOUND DRAIN AND COVER

(71) Applicant: Shree Devi Visaria, Houston, TX (US)

(72) Inventor: Shree Devi Visaria, Houston, TX (US)

(21) Appl. No.: 15/453,631

(22) Filed: Mar. 8, 2017

Related U.S. Application Data
(60) Provisional application No. 62/375,317, filed on Aug. 15, 2016.

(51) Int. Cl. A61M 1/00; A61F 13/00; A61M 27/00

(52) U.S. Cl. CPC A61M 1/0088; A61M 27/00; A61M 1/0086; A61F 13/00068; A61M 1/0072; A61M 2205/0205; A61M 2210/1021

(57) ABSTRACT

A wound drain that includes a drain tube configured to drain fluid from an interior cavity of a wound, a fluid wicking bandage configured to wick fluid from a surface of the wound, and a suction flange configured to pull fluid from the drain tube and the fluid wicking bandage into a suction flange tube. The suction flange tube is configured to be connected to a vacuum source.

### CROSS REFERENCE TO RELATED APPLICATIONS

The present application is a non-provisional application that claims the benefit of, and the priority from, U.S. Provisional Patent Application No. 62/375317 having a title of “Wound Drain and Cover,” filed Aug. 15, 2016, which is incorporated entirely herein.

### FIELD

The present invention relates, generally, to systems and methods usable for draining and protecting wounds. Particularly, the wounds may result from surgical incisions into a patient's abdominal cavity, and negative pressure drain(s) may be needed to siphon fluid from deep within and around the incision. In addition, surface negative pressure allows for superficial incision drainage and reduces tension on wound edges thereby facilitating the healing process.

### BACKGROUND

Surgeons and other physicians providing post-op care to surgery patients have a need to quickly, efficiently, and cleanly drain the fluid formed within the body and around the incision. To remove this accumulation of fluid, these physicians have used forms of deep tissue drains and bandages for minimizing the problems in the healing process caused by the accumulation of the fluid.

In some surgery patients, fluid can develop below the surface of an incision, causing a seroma to form. This fluid develops due to leakage of interstitial fluid that may drain for days or weeks after the surgery is completed. The subcutaneous tissue below the skin incision tends to seep fluid that needs to be directed away from the general incision area and out of the body.

The problem of removing this accumulation of fluid is particularly pronounced in patients that have a significant amount of tissue (e.g., adipose tissue, etc.) below the skin surface. For these patients, the amount of serous fluid draining into the wound may exceed the body's own lymphatic drainage capabilities and/or evacuation limits of the existing drain systems and techniques. When this happens, the skin above the wound may become saturated by fluid that drains through the wound to the surface of the skin. That is, some serous fluid (or other fluids) may seep passed the patient's sutures, staples, and/or other wound closing treatments, to stagnate on the patient's skin. As such, this extra, stagnant fluid can cause severe discomfort to the patient, maceration of the patient's skin, and/or significantly higher risk of infection to the patient. Therefore, a need exists for apparatus and methods usable to remove fluid, including an accumulation of fluid, from within the body and around the surface of a wound (e.g., incision) in a sufficient amount that leaves the patient's skin, surrounding the wound, dry and sterile (e.g., free from bacterial growth and infection). In addition, a need exists for a deep tissue drain, which can apply a negative pressure therapy to the primary closed incision, for minimizing the problems in the healing process caused by the accumulation of fluid formed from a wound (e.g., surgical incision).

In some instances, bandages have been used to absorb some of the fluid leaking from around the surface of the wound and the patient's skin. These bandages, however, can become fluid soaked and contribute to the potential for infection. To protect against possible infection from fluid-soaked bandages, a wound may need a change of bandages every couple of hours, especially during the time just after surgery is completed. In many instances, the patient lacks expertise to change the bandages, necessitating a professional to be summoned regularly for the changing of these bandages, which increases the burden on the medical staff, and increases the risks of late or forgotten changes of the bandages. Accordingly, a further need exists for apparatus and methods usable to decrease the time and resources needed to assist these patients, who have surgical wounds, for quickly and efficiently draining the fluid formed within the body and around the surface of the wound (e.g., incision) and for maintaining dry and sterile bandaging to reduce the risk of infection and promote the patient's healing process.

The present embodiments meet all of these needs.

### SUMMARY

The disclosed embodiments include a wound drain having a drain tube configured to drain fluid from an interior cavity of a wound, a fluid wicking bandage configured to wick fluid from a surface of the wound, and a suction flange configured to separately pull fluid from the drain tube and the fluid wicking bandage into a suction flange tube, wherein the suction flange tube is configured to be connected to a vacuum source.

In certain embodiments, the drain tube includes a collar configured to rest on the surface of the wound to prevent the drain tube from slipping into the wound, a drain incision, or combinations thereof. The collar may also include perforations configured to enable fluid to pass from the surface of the wound to the fluid wicking bandage. In certain embodiments, the suction flange includes a plenum between the fluid wicking bandage and the suction flange tube.

In certain embodiments, the wound drain includes a hole between the plenum and the drain tube. The hole is configured to provide a pressure differential between the plenum and the drain tube. In certain embodiments, the suction flange includes an opening that directly contacts the fluid wicking bandage and a hole that directly contacts the deep tissue drain. In certain embodiments, the vacuum source includes an electronic pump, a vacuum bulb, or combinations thereof. In certain embodiments, the suction flange is configured to absorb fluid from the entire area of the fluid wicking bandage. Also, the fluid wicking bandage may include an antimicrobial layer.

The disclosed embodiments may also include a method of placing a wound drain in a patient. The method may include placing a deep drain tube within a wound below a skin surface of the patient and above a fascia of the patient, connecting the drain tube to a suction flange, wherein the suction flange is integrated with a fluid wicking bandage, placing the fluid wicking bandage on the skin surface to cover the wound, securing a seal over the fluid wicking bandage, and connecting the suction flange to a vacuum source. The vacuum source may be configured to pull fluid from the deep drain tube and the fluid wicking bandage through a single suction flange tube.

In certain embodiments, placing the deep drain tube includes cutting a drain incision a distance away from the wound, and drawing the drain through the drain incision into the wound, or placing the deep drain tube includes cutting the drain tube to a length based on a size of the wound. In certain embodiments, securing the seal includes monitoring the fluid wicking bandage for decompression after the vacuum source is connected and turned on. In certain embodiments, the method may include monitoring a reservoir in the vacuum source, and changing the reservoir of the vacuum source without removing the vacuum pressure from the suction flange.

The disclosed embodiments may also include a system for draining fluid from a patient. The system may include a vacuum source configured to maintain a vacuum pressure, a reservoir connected to the vacuum source and configured to hold an amount of fluid, a drain tube configured to drain fluid from a first location of a wound, a fluid wicking bandage configured to absorb fluid from a second location of a wound, and a suction flange connected to the vacuum source and configured to convey the vacuum pressure to both the drain tube and the fluid wicking bandage.

In certain embodiments, the reservoir is configured to switch with an empty replacement reservoir. In certain embodiments, the suction flange includes a plenum between the fluid wicking bandage and the suction flange tube. The system may also include a hole between the plenum and the drain tube, wherein the hole is configured to provide a pressure differential between the plenum and the drain tube. In certain embodiments, the fluid wicking bandage includes an antimicrobial layer. And, the system may include a seal configured to seal the vacuum pressure between the fluid wicking bandage and a skin surface of the patient.

### BRIEF DESCRIPTION OF THE DRAWINGS

FIG. 1 depicts a perspective view of a patient having a surgical wound from an abdominal-cavity operation.

FIG. 2 illustrates a perspective view of a patient with an embodiment of a negative pressure wound drain placed within the wound.

FIG. 3 illustrates a cross-sectional side view of an embodiment of the negative pressure wound drain that has been placed within the wound of the patient.

FIG. 4 is a cross-sectional side view of an embodiment of the flange having a vacuum bulb that maintains the vacuum pressure to drain the wound.

### DETAILED DESCRIPTION OF THE EMBODIMENTS

Before explaining selected embodiments of the present invention in detail, it is to be understood that the present invention is not limited to the particular embodiments described herein and that the present invention can be practiced or carried out in various ways.

The present invention relates, generally, to a system usable to provide negative pressure wound therapy for primary skin closures and deep tissue drains. The disclosed embodiments are particularly beneficial for patients with conditions that may impair wound healing. Such conditions may include obesity, diabetes, age, steroid use, immunosuppression, and impaired oxygenation. The disclosed embodiments may be used with clean, primary incision closures for any type of laparotomy, large suprafascial procedures (e.g., abdominoplasty), and large, deep soft tissue incisions.

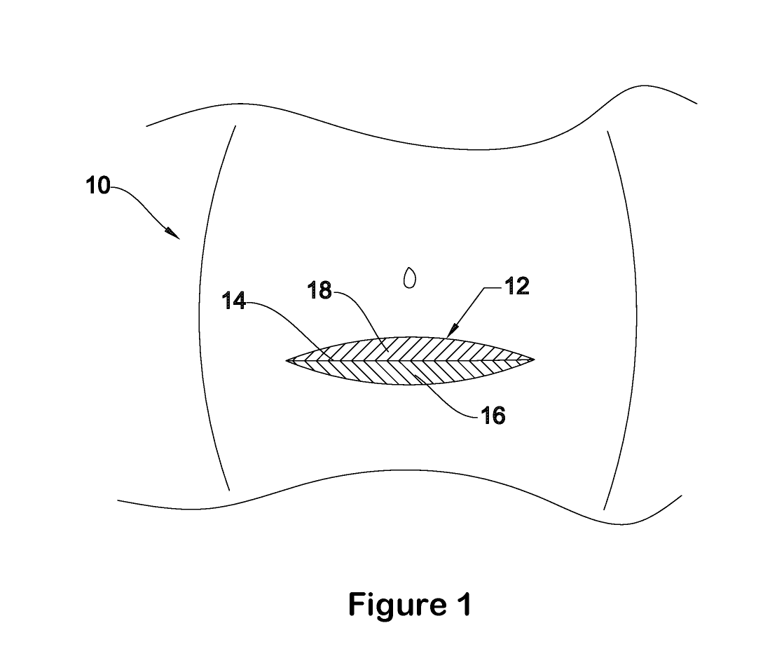

FIG. 1 depicts a perspective view of a patient body 10 having a surgical wound 12 from an abdominal-cavity operation. The disclosed embodiments below may be addressed, in particular, to operations involving abdominal wounds, but the embodiments may be understood to be used in any deep-tissue procedure in any location, or orientation, on the patient's body 10. Additionally, while embodiments of the disclosed invention may be used in subfascial procedures, the description will focus on draining operations after a fascia 14 is closed, or has not been cut. FIG. 1 illustrates the wound 12 through a skin layer 16, a subcutaneous layer 18, to the fascia 14. As explained above, the subcutaneous layer 18 can produce fluid for days or weeks after the surgery, and during the healing process. As shown and described, the disclosed embodiments of the deep tissue drain, for applying a negative pressure therapy to the wound or primary closed incision, will work for many or all different types of surgical incisions and other wounds as the skin 16 and the subcutaneous layer 18 may vary in thickness and fluid production.

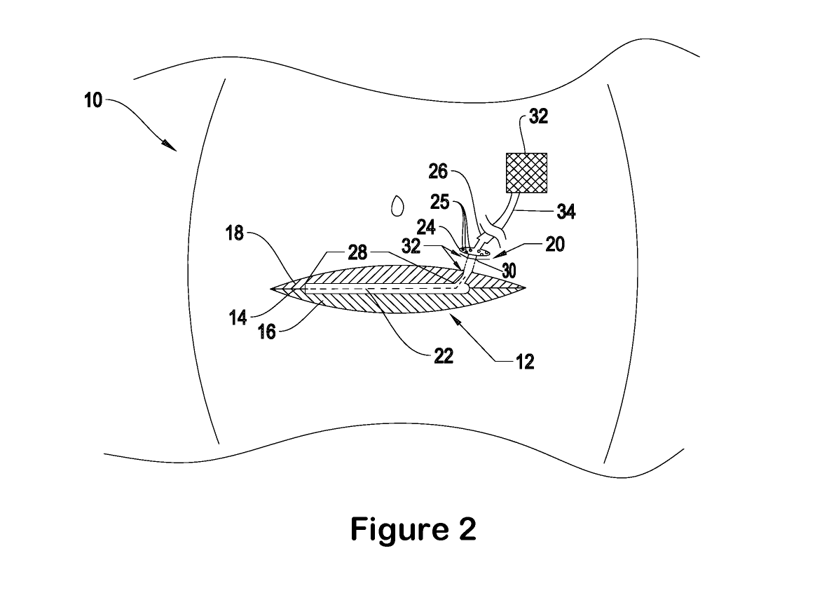

FIG. 2 illustrates a perspective view of the patient' body 10 with a deep tissue, negative pressure wound drain 20 placed within the wound 12 (e.g., surgical incision). The deep tissue, negative pressure wound drain 20, as shown, includes a drain tube 22, a collar 24, and a fitting flange 26, which can be connected to a suctioning device or vacuum that is usable to drain fluid from the wound 12.

The drain tube 22 may be flat or round, and fenestrated along an internal section 28 of the drain tube 22 to better enable the fluid to be pulled from the wound 12. Rather than having the drain tube 22 run directly out of the wound 12, the drain tube 22 may pass through a drain incision 30 that is cut a distance 32 away from the wound 12. The drain incision 30 may be placed so that the drain tube 22 is away from the wound 12 on the surface of the skin 16, but enters the wound 12 at some point before sitting at the bottom of the wound 12. The drain incision 30 can help to ensure that the deep tissue, negative pressure wound drain 20 does not shift after installation, which can help to enable the wound 12 to properly heal. The distance 32 of the drain incision 30 from the wound 12 may be determined by the surgeon performing the operation and according to the patient's skin properties. For example, the drain incision 30 may be located approximately 2 cm (0.787 inches) from the wound 12. Then, after forming the drain incision 30, the drain tube 22 may be pulled through the drain incision 30 and trimmed so that the internal section 28 of the drain tube 22 matches the incision length of the wound 12.

To prevent the deep tissue, negative pressure wound drain 20 from slipping further into the drain incision 30, or from slipping out of the wound 12 completely, deep tissue, negative pressure wound drain 20 may include a collar 24 that can lay flat against the patient's skin. After surgery, the collar 24 can be covered by a foam covering, that includes a flange with the drain tube 22. The drain tube hold the collar in place and protect the wound 12. The collar 24 may include perforations 25 to allow fluid from the wound 12 to pass from the surface of the wound 12 through to the foam covering for collection and removal.

After the drain tube 22 directs deep tissue fluid from the wound 12, the fluid is drawn through the fitting 26 and into a vacuum source 32. As explained in detail below, the vacuum source 32 may include mechanical, electronic, or other vacuum sources to maintain a pressure differential. In an embodiment, the pressure differential may be between 0.0 and 170.0 mm Hg. Furthermore, the vacuum source 32 may be located adjacent to the patient's body 10, or may be remotely located and connected to the wound drain 20 by a connecting tube 34.

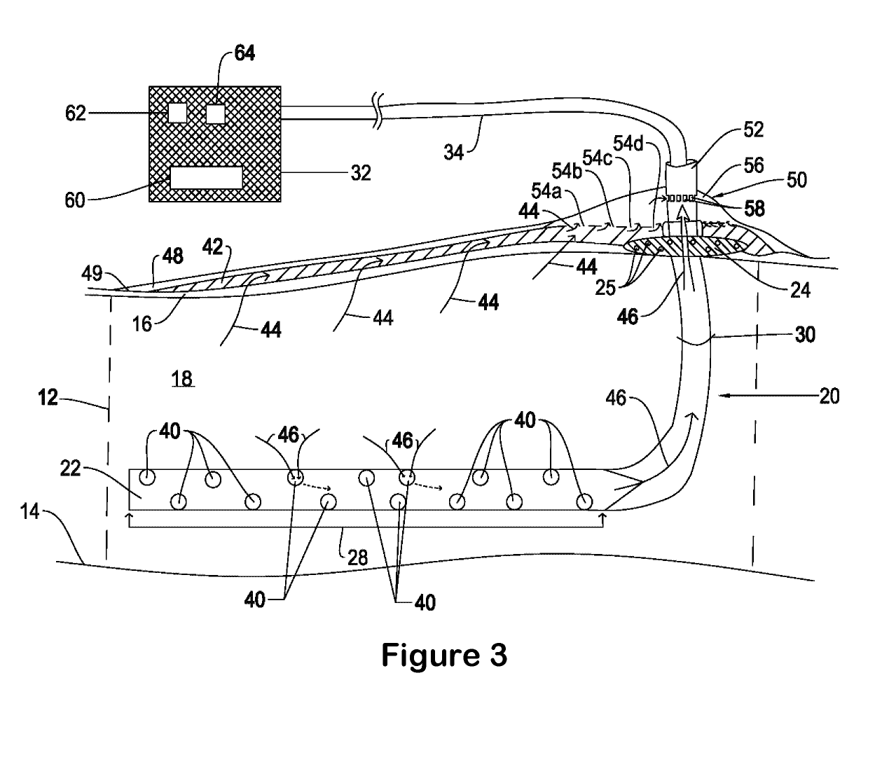

FIG. 3 illustrates a cross-sectional side view of an embodiment of the deep tissue, negative pressure wound drain 20 that has been placed within the wound 12 of the patient's body 10. The drain tube 22, as illustrated, has been pulled through the drain incision 30 and placed within the wound 12 at a deep location near the fascia 14. As shown, the drain tube 22 includes a fenestrated internal section 28 that is perforated by holes 40 to allow deep tissue fluid 46 to pass through the holes 40 and into the drain tube 22. In the embodiment of FIG. 3, the deep tissue fluid 46 drains into the drain tube 22, toward the connecting tube 34, and eventually to the vacuum source 32.

The illustrated embodiment of FIG. 3 also includes a foam covering 42 for absorbing surface fluid 44 that is seeping through the wound 12 at the surface of the skin 16. As outlined above, the foam covering 42 may absorb superficial seepage (surface fluid 44), provide an intermediary barrier for patient comfort, and also aid in maintaining negative pressure over the wound 12. A seal 48, can be placed over and around the foam covering 42, to ensure that the surface fluid 44 does not leak beyond the foam covering 42. The seal 48 may be made of plastic, rubber, silicone polymer, or other pliable material, and the seal 48 can include an adhesive rim 49 that can adhere to the patient's skin 16, such that pressure is not lost due to incoming air leaks. The foam covering 42 may include features, such as channels, mesh, variations in foam density, and variations in foam thickness to ensure a constant suction from the extremities of the foam covering 42 toward a flange 50. For example, the foam covering 42 may have multiple layers, such as a mesh layer, to absorb or promote movement of the surface fluid 44 away from the skin 16, and toward the flange 50.

In certain embodiments, the flange 50 can be the housing component for the flange tube 52, and the structure by which the surface fluid 44 may be siphoned from the foam covering 42. Within the housing of the flange 50, the flange tube 52 interfaces with drain tube 20 and exiting tubing 34. Flange tube 52 has holes 58 that allow surface fluid 44 to meet the higher volume deep drain fluid 46. The flange 50 minimizes or prevents leakage of deep drain fluid 46 into the foam covering 42. The combined fluids (44 and 46) may then exit together to connection tube 34. The suction flange tube 52 can ensure that a constant and consistent vacuum pressure is maintained for both the drain tube 22 and the foam covering 42. The flange 50 interfaces with a “push on” or “snap on” leak resistant fitting on drain tube 22.

The flange 50 may include several openings 54 that are usable to pull the surface fluid 44 from the foam covering 42 and into a plenum 56. The plenum 56 may include an internal volume that conveys the vacuum pressure evenly to the openings 54 in the flange 50. The openings 54 may vary in size depending on the expected vacuum pressure and the amount of surface fluid 44 being drawn from the foam covering 42. For example, if the amount of surface fluid 44 is large, the openings 54 may be large to allow more surface fluid 54 into the plenum 56. Alternatively, the size of the openings 54 may vary based on the relative location of the openings 54 from the flange tube 52. That is, the openings 54c, 54d that are closer to the flange tube 52 may be smaller than the openings 54a, 54b that are farther from the flange tube 52, or vice versa. In certain embodiments, the openings 54 are each small when compared to the flange tube 52. This reflects the higher amount of deep tissue fluid 46 that passes through the flange tube 52, when compared to the relatively smaller amount of surface fluid 44 that passes through the flange 50 and the plenum 56.

In certain additional or alternative embodiments, the flange 50 may include holes 58 between the plenum 56 and the flange tube 52 to allow the surface fluid 44 to flow into the flange tube 52 with the deep tissue fluid 46. The holes 58 may vary in size, shape, orientation, or other characteristics, and these dimensions can vary or be adjusted based upon the amount of surface fluid 44 that must be drained relative to the amount of deep tissue fluid 46 that must be drained from the patient's body 10. For example, with regard to wounds 12 that are sealed tightly at the skin surface 16, it may be expected that the amount of surface fluid 44 would be low; and thus, the holes 58 would be smaller, fewer, or some combination thereof. With regard to wounds 12 with a large amount of expected surface fluid 44, the holes 58 would be larger, more plentiful, or some combinations thereof.

The holes 58 may also be shaped to customize a pressure within the plenum 56. For example, the holes 58 may be relatively smaller to ensure a slight pressure differential between the plenum 56 and the flange tube 52. In this instance, the pressure differential would ensure that the surface fluid 44 does not seep into the flange tube 52 and down into the drain tube 22. Additionally or alternatively, the holes 58 may be cupped on the inside of flange tube 52 to minimize leakage of fluid into the foam covering 42, especially during loss of negative pressure when emptying or replacing the reservoir 60 or with patient movement. Additionally or alternatively, the size of the flange tube 52 may be larger or smaller, have a varied shape, or some combination thereof, to adjust the ratio of surface fluid 44 to deep tissue fluid 46 that is drained by the deep tissue negative pressure wound drain 20 to the vacuum source 32. The vacuum source 32 may include a reservoir 60, indicator lights 62, and control switches 64 (e.g., buttons) for operating and controlling the vacuum source 32. The reservoir 60 can be used to store the fluid, including the surface fluid 44 and the deep tissue fluid 46, that is drained from the deep tissue negative pressure wound drain 20. The reservoir can include hash marks or other indicators to show the amount of fluid 44, 46 that has been drained. The indicator lights 62 of the vacuum source 32 may show the amount of fluid 44, 46 that has been drained as well, and may indicate whether the reservoir 60 is nearing the limit of its capacity, among providing other indications. The control switches 64 may be used to adjust the vacuum pressure, turn on the vacuum source 32, or perform other control operations for the vacuum source 32.

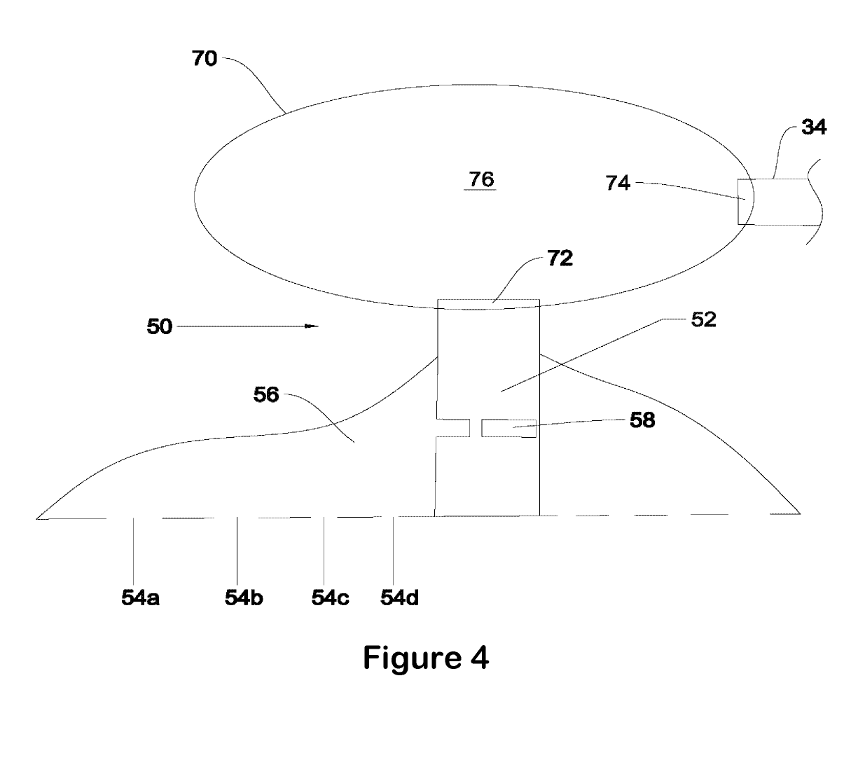

FIG. 4 is a cross-sectional side view of an embodiment of the flange 50 having a vacuum bulb 70 that maintains a negative or vacuum pressure to drain the wound 12. The flange 50 has openings 54 that can be usable to drain the surface fluid 44 from the foam covering 42, as described above. In the illustrated embodiment, the openings 54a and 54b, which are farther from the flange tube 52, are larger than the openings 54c and 54d that are closer to the flange tube 52. The illustrated embodiment also shows an alternative embodiment for the holes 58 of the flange tube 52. In this embodiment, the holes 58 are wider to enable more surface fluid 44 to flow from the plenum 56 into the flange tube 52.

After entering the flange tube 52, the surface fluid 44 can combine or mix with the deep tissue fluid 46 and the combination of the fluids 44, 46 can drain into the vacuum bulb 70. To maintain the vacuum pressure, the vacuum bulb 70 can include, in an embodiment, an intake valve 72 and a release valve 74. To start the process, an operator manually squeezes the vacuum bulb 70, which compresses an interior volume 76 of the vacuum bulb 70. During the squeeze, the intake valve 72 remains sealed and the release valve 74 unstops (opens) to enable the air, fluid, or combinations thereof, to evacuate through the connection tube 34.

While various embodiments of the present invention have been described with emphasis, it should be understood that within the scope of the appended claims, the present invention might be practiced other than as specifically described herein.

1. A wound drain, comprising:
a drain tube configured to drain fluid from an interior cavity of a wound;
a fluid wicking bandage configured to wick fluid from a surface of the wound; and
a suction flange configured to separately pull fluid from the drain tube and the fluid wicking bandage into a suction flange tube, wherein the suction flange tube is configured to be connected to a vacuum source.

2. The wound drain of claim 1, wherein the drain tube comprises a collar configured to rest on the surface of the wound to prevent the drain tube from slipping into the wound, a drain incision, or combinations thereof.

3. The wound drain of claim 2, wherein the collar comprises perforations configured to enable fluid to pass from the surface of the wound to the fluid wicking bandage.

4. The wound drain of claim 1, wherein the suction flange comprises a plenum between the fluid wicking bandage and the suction flange tube.

5. The wound drain of claim 4, further comprising a hole between the plenum and the drain tube, wherein the hole is configured to provide a pressure differential between the plenum and the drain tube.

6. The wound drain of claim 1, wherein the suction flange comprises an opening that directly contacts the fluid wicking bandage and a hole that directly contacts the deep tissue drain.

7. The wound drain of claim 1, comprising the vacuum source, wherein the vacuum source comprises an electronic pump, a vacuum bulb, or combinations thereof.

8. The wound drain of claim 1, wherein the suction flange is configured to absorb fluid from the entire area of the fluid wicking bandage.

9. The wound drain of claim 1, wherein the fluid wicking bandage comprises an antimicrobial layer.

10. A method of placing a wound drain in a patient, comprising
placing a deep drain tube within a wound below a skin surface of the patient and above a fascia of the patient;
connecting the drain tube to a suction flange, wherein the suction flange is integrated with a fluid wicking bandage;
placing the fluid wicking bandage on the skin surface to cover the wound;
securing a seal over the fluid wicking bandage; and
connecting the suction flange to a vacuum source, wherein the vacuum source is configured to pull fluid from the deep drain tube and the fluid wicking bandage through a single suction flange tube.

11. The method of claim 10, wherein placing the deep drain tube comprises cutting a drain incision a distance away from the wound, and drawing the drain through the drain incision into the wound.

12. The method of claim 10, wherein placing the deep drain tube comprises cutting the drain tube to a length based on a size of the wound.

13. The method of claim 10, wherein securing the seal comprises monitoring the fluid wicking bandage for decompression after the vacuum source is connected and turned on.

14. The method of claim 10, comprising, monitoring a reservoir in the vacuum source, and changing the reservoir of the vacuum source without removing the vacuum pressure from the suction flange.

15. A system for draining fluid from a patient, comprising:
a vacuum source configured to maintain a vacuum pressure;
a reservoir connected to the vacuum source and configured to hold an amount of fluid;
a drain tube configured to drain fluid from a first location of a wound;
a fluid wicking bandage configured to absorb fluid from a second location of a wound; and
a suction flange connected to the vacuum source and configured to convey the vacuum pressure to both the drain tube and the fluid wicking bandage.

16. The system of claim 15, wherein the reservoir is configured to switch with an empty replacement reservoir.

17. The system of claim 15, wherein the suction flange comprises a plenum between the fluid wicking bandage and the suction flange tube.

18. The system of claim 17, further comprising a hole between the plenum and the drain tube, wherein the hole is configured to provide a pressure differential between the plenum and the drain tube.

19. The system of claim 15, wherein the fluid wicking bandage comprises an antimicrobial layer.

20. The system of claim 15, comprising a seal configured to seal the vacuum pressure between the fluid wicking bandage and a skin surface of the patient.
